(19) United States
(12) Patent Application Publication
(10) Pub. No.: US 2026/0209865 A1
(43) Pub. Date: Jul. 23, 2026

(54) DETERMINATION OF SPECIES, DETECTION OF DISEASE AND PATHOGENICITY DETECTION METHOD IN EUKARYOTIC EXOSOMES AND BACTERIAL OUTER MEMBRANE VESICLES

(71) Applicant: BILKENT UNIVERSITESI ULUSAL NANOTEKNOLOJI ARASTIRMA, Ankara (TR)

(72) Inventors: Fatih INCI, Ankara (TR); Nedim HACIOSMANOGLU, Ankara (TR)

(73) Assignee: BILKENT UNIVERSITESI ULUSAL NANOTEKNOLOJI ARASTIRMA, Ankara (TR)

(21) Appl. No.: 19/142,453

(22) PCT Filed: Nov. 17, 2023
(86) PCT No.: PCT/TR2023/051341

§ 371 Date: Jun. 23, 2025

(30) Foreign Application Priority Data
Dec. 30, 2022 (TR) 2022/021664

(51) Int. Cl. C12Q 1/689; B01L 3/00; C12Q 1/6844; C12Q 1/6888

(52) U.S. Cl. CPC C12Q 1/689; B01L 3/502707; C12Q 1/6844; C12Q 1/6888; B01L 2200/0647; B01L 2200/12; B01L 2300/0681; B01L 2300/12; B01L 2400/0487

(57) ABSTRACT

The present invention relates to a method for performing determination of species, realizing disease and pathogenicity detection in eukaryotic exosomes and bacterial outer membrane vesicles of isothermal nucleic acid amplification methods.

### TECHNICAL FIELD

The present invention relates to a method for performing determination of species, realizing disease and pathogenicity detection in eukaryotic exosomes and bacterial outer membrane vesicles of isothermal nucleic acid amplification methods.

### BACKGROUND OF THE INVENTION

Bacterial outer membrane vesicles (BOMVs) and eukaryotic exosomes (EEs) are fat vesicles of 30-200 nm in size which are formed by the cell upon living cells proliferate as a result of different stress and disease factors. During their formation from the membrane, these vesicles also include in different cellular elements and therefore, they carry many molecules such as proteins, nucleic acids and other small molecules which can provide insight into the internal environment of the cell. Nucleic acids are molecules present in all living cells and carry the information necessary for carrying out vital activities. With BOMVs and EEs, it is possible to have information about the nucleic acids included in the cell without disrupting the cell integrity and this creates an important target for non-invasive disease detection. In addition to this, it has been reported in the literature that BOMVs and EEs can circulate freely in body fluids and this provides convenience in terms of sampling. On the other hand, isolation of EEs and BOMVs carried out from body fluids and medium samples requires advanced and expensive equipment (ultra-fast centrifugation, etc.) when it is performed with a centrifuge-based isolation scheme that is used as the classical method in the literature and this makes BOMV and EE-based disease/pathogenicity detection long and laborious. Even in this case, the purity of BOMVs and EEs obtained by conventional methods remains at low level.

Quantitative Real-Time Polymerase Chain Reaction (QR-PCR), which is used as the “gold standard”, is the leading nucleic acid detection method used in the characterization of isolated BOMVs and EEs. In this method, amplification of nucleic acids can be monitored in real-time and by means of nucleic acid dyes used and this process can be carried out specifically for the target region via region-specific amplifying nucleic acids. However, due to the high-cost infrastructure and the experienced personnel requirement of this method, it is not possible to apply it in the field and to yield result in a short time. Isothermal nucleic acid amplification methods (INAA) are nucleic acid amplification methods which can be performed at a single temperature, unlike methods developed alternatively and require temperature cycles such as QR-PCR. By means of to these features thereof, INAA methods have the potential of applicability to the field and it can be performed by any user with low instrumentation requirements at low level. Again, the reaction monitoring required to analyze the results in this method can be carried out via methods which do not require expensive nucleic acid probes.

The need for advanced and expensive equipment for BOVM and EE isolation methods, which are used as the classical method, and also the fact that the sample purity is low and the QR-PCR method, which is used as the gold standard for the detection of nucleic acids in isolated samples, is quite high cost indicate that there is need for a new detection method.

The United States patent document no. US2022098575, an application included in the state of the art, discloses a system and method for obtaining genetic information from nucleic acids in ultra-low amounts of biological samples.

### SUMMARY OF THE INVENTION

An objective of the present invention is to realize a method for performing determination of species, realizing disease and pathogenicity detection in eukaryotic exosomes and bacterial outer membrane vesicles of isothermal nucleic acid amplification methods.

### DETAILED DESCRIPTION OF THE INVENTION

“Determination of Species, Detection of Disease and Pathogenicity Detection Method in Eukaryotic Exosomes and Bacterial Outer Membrane Vesicles” realized to fulfil the objective of the present invention is shown in the figures attached, in which:

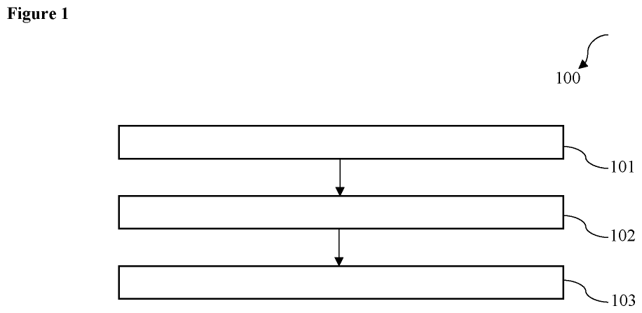

FIG. 1 is a flow chart of the inventive method.

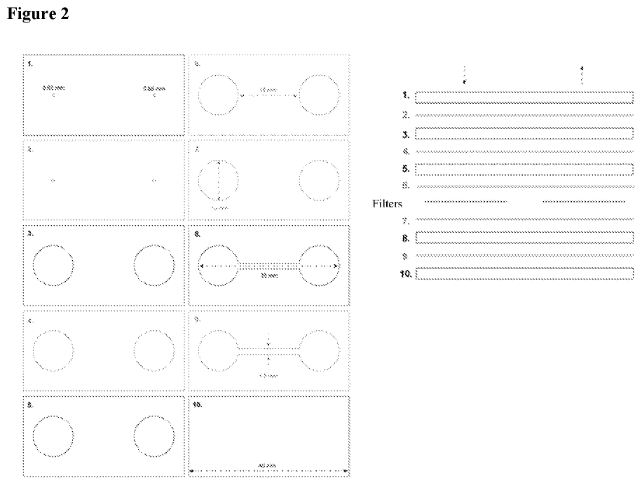

FIG. 2 is a view of the layers of the microfluidic filtration chip obtained in the inventive method.

The components illustrated in the figures are individually numbered, where the numbers refer to the following:
100. Method

The inventive method (100) for performing determination of species, realizing disease and pathogenicity detection in eukaryotic exosomes and bacterial outer membrane vesicles of isothermal nucleic acid amplification methods; comprises steps of:
preparing a microfluidic filtration chip (101);
providing a sample, for which determination of species will be performed and detection of disease and pathogenicity will be realized, to a microfluidic filtration chip (102);
performing determination of species, detection of disease and pathogenicity upon isothermal nucleic acid test is applied to a sample obtained from a microfluidic filtration chip (103).

In the step of preparing a microfluidic filtration chip (101) of the inventive method (100); five polymer material (PMMA) layers which form the 1^{st}, 3^{rd}, 5^{th}, 8^{th} and 10^{th} layers—preferably 2 mm thick—to be used in producing microfluidic chip, have the same size as the polymer layer and five double-sided adhesive (DSA, 50 μm thick) layers which form the 2^{nd}, 4^{th}, 6^{th}, 7^{th} and 9^{th} layers are cut by means of a laser cutter. On the 1^{st} and 2^{nd} layers, sample inlet and outlet holes are created in sizes such that capillary tube tips can enter. On the 3^{rd}, 4^{th}, 5^{th}, 6^{th}, 7^{th} and 8^{th} layers, holes larger than the inlet and outlet holes are created such that they will contain the sample inlet and outlet holes in their center. A passageway interconnecting the holes between the holes connecting on the 8^{th} and 9^{th} layers is created. All layers are superimposed from 1 to 10 respectively and two filters with variable scales of 10-200 nanometers are placed between the 6^{th} and the 7^{th} layers. Layers, which are superimposed such that filters will be provided between thereof, are compressed by means of a compression mechanism. Capillary tubes are fixed with resin in the inlet and outlet holes of the 1^{st} layer. A bioreactor chamber and a bioreactor attachment, which is obtained by placing a filter and magnetic stirrer between two plates with holes on them, are joined to the microfluidic chip. A LED lamp and a light sensor, which will be used for measuring the growth of the bacterial culture, are attached to the sides of the bioreactor attachment.

In the step of providing a sample, for which determination of species will be performed and detection of disease and pathogenicity will be realized, to a microfluidic filtration chip (102) of the inventive method (100); samples are obtained directly by being received from patients in a liquid form or after the liquid is combined with a sterile medium in the bioreactor section of the microfluidic chip and then the medium is homogeneously mixed with a magnetic stirrer. The sample is combined with the capillary tube located at the inlet hole of the microfluidic chip. A sample which is passed through the capillary tube with a volume of 1-2 mL and at a rate of 40 μL/minute by means of a syringe pump, a peristaltic pump or any other pump that can be used in liquid manipulation, is introduced into the microfluidic chip. Upon the sample is introduced into the microfluidic chip, filtering takes place during the liquid flow in the chip and an isolated vesicle (isolate or eluant) is obtained from the outlet end of the chip, which is separated from the sample's residual materials (debris), i.e. biological molecule separation.

In the step of performing determination of species, detection of disease and pathogenicity upon isothermal nucleic acid test is applied to a sample obtained from a microfluidic filtration chip (103) of the inventive method (100); a strain determination is realized by transferring the isolated vesicle obtained from the microfluidic chip, to an isothermal nucleic acid amplification reaction (nucleic acid amplification reaction occurring at single-temperature such as LAMP, RPA or etc.) containing species-specific primers. The target nucleic acids used in this step arise exclusively from the isolated vesicles.

For biological molecule separation, after the cells are precipitated at 10.000 g and filtered with a filter in a 0.2 μm pore diameter, the remaining medium supernatant is passed through a microfluidic chip system. In this system, the inlet part of the microfluidic chip is attached to the syringe wherein the sample supernatant is included and the outlet part is collected in a 2 mL Eppendorf. Upon the sample is passed in a volume of 1 or 2 mL at a rate of 40 μL/min, the system wash is performed with 1 or 2 mL of PBS. The eluant containing the obtained and isolated BOMVs is used for isothermal nucleic acid testing. In addition, for on-chip growth, a low amount of bacterial cells are injected into the chamber in the microchip through the cell inlet and thus it is ensured that they grow by creating a dynamic culture environment and they send BOMV out of the filtration system in the process.

LAMP assay-which is selected as a model for isothermal reactions (in accordance with the protocols of NEB company)—10X Isothermal Reaction Solution, MgSO4 (100 mM), dNTP Mixture (10 mM), LAMP Primers stock (10X), Bst 2.0 WarmStart DNA Polymerase (NEB, 8/Reaction) are used for the species-specific detection of isolated BOMVs and WarmStart RTx Reverse Transcriptase (NEB, 0.5 μL/Reaction) is used for RNA detection. Whereas for qPCR test, the reaction solution (100 mM KCI, 160 mM (NH4)2SO4, 20 mM MgSO4, 200 mM Tris-HCl (pH: 8.8), 1% Triton X-100, 1 mg/mL Bovine Serum Albumin, 10 Mm dNTP) is used to monitor the PFU/Psp DNA Polymerase (BIOSWISSTEC) enzyme to amplify the target DNA region. Analytical monitoring of these two reactions is performed in the step 103 of the method (100) by using double-stranded DNA dye (NEB, LAMP Reaction Dye, Thermo Scientific, SYBR Green) or pH-sensitive dyes (Phenol Red) or Magnesium dye (Hydroxynaphtol Blue) or turbidity test. The follow-up time for the related LAMP reactions is 45 minutes, whereas the follow-up time is calculated to be approximately 2 hours for qPCR, depending on the cycle time and the performance of the instrument (BioRad).

Within these basic concepts; it is possible to develop various embodiments of the inventive “Determination of Species, Detection of Disease and Pathogenicity Detection Method (100) in Eukaryotic Exosomes and Bacterial Outer Membrane Vesicles”; the invention cannot be limited to examples disclosed herein and it is essentially according to claims.

1. A method (100) for performing determination of species, realizing disease and pathogenicity detection in eukaryotic exosomes and bacterial outer membrane vesicles of isothermal nucleic acid amplification methods; characterized in that it comprises steps of:
preparing a microfluidic filtration chip (101);
providing a sample, for which determination of species will be performed and detection of disease and pathogenicity will be realized, to a microfluidic filtration chip (102);
performing determination of species, detection of disease and pathogenicity upon isothermal nucleic acid test is applied to a sample obtained from a microfluidic filtration chip (103).

2. A method (100) according to claim 1; characterized in that in the step of preparing a microfluidic filtration chip (101); five polymer material (PMMA) layers which form the 1^{st}, 3^{rd}, 5^{th}, 8^{th} and 10^{th} layers—preferably 2 mm thick—to be used in producing microfluidic chip, have the same size as the polymer layer and five double-sided adhesive (DSA, 50 μm thick) layers which form the 2^{nd}, 4^{th}, 6^{th}, 7^{th} and 9^{th} layers are cut by means of a laser cutter.

3. A method (100) according to claim 2; characterized in that in the step of preparing a microfluidic filtration chip (101); on the 1^{st} and 2^{nd} layers, sample inlet and outlet holes are created in sizes such that capillary tube tips can enter.

4. A method (100) according to claim 2; characterized in that in the step of preparing a microfluidic filtration chip (101); on the 3^{rd}, 4^{th}, 5^{th}, 6^{th}, 7^{th} and 8^{th} layers, holes larger than the inlet and outlet holes are created such that they will contain the sample inlet and outlet holes in their center.

5. A method (100) according to claim 2; characterized in that in the step of preparing a microfluidic filtration chip (101); a passageway interconnecting the holes between the holes connecting on the 8^{th} and 9^{th} layers is created.

6. A method (100) according to claim 2; characterized in that in the step of preparing a microfluidic filtration chip (101); all layers are superimposed from 1 to 10 respectively and two filters with variable scales of 10-200 nanometers are placed between the 6^{th} and the 7^{th} layers.

7. A method (100) according to claim 1; characterized in that in the step of preparing a microfluidic filtration chip (101); layers, which are superimposed such that filters will be provided between thereof, are compressed by means of a compression mechanism.

8. A method (100) according to claim 2; characterized in that in the step of preparing a microfluidic filtration chip (101); capillary tubes are fixed with resin in the inlet and outlet holes of the 1^{st} layer.

9. A method (100) according to claim 2; characterized in that in the step of preparing a microfluidic filtration chip (101); a bioreactor chamber and a bioreactor attachment, which is obtained by placing a filter and magnetic stirrer between two plates with holes on them, are joined to the microfluidic chip.

10. A method (100) according to claim 1; characterized in that in the step of preparing a microfluidic filtration chip (101); a LED lamp and a light sensor, which will be used for measuring the growth of the bacterial culture, are attached to the sides of the bioreactor attachment.

11. A method (100) according to claim 1; characterized in that in the step of providing a sample, for which determination of species will be performed and detection of disease and pathogenicity will be realized, to a microfluidic filtration chip (102); samples are obtained directly by being received from patients in a liquid form or after the liquid is combined with a sterile medium in the bioreactor section of the microfluidic chip and then the medium is homogeneously mixed with a magnetic stirrer.

12. A method (100) according to claim 11; characterized in that in the step of providing a sample, for which determination of species will be performed and detection of disease and pathogenicity will be realized, to a microfluidic filtration chip (102); the sample is combined with the capillary tube located at the inlet hole of the microfluidic chip.

13. A method (100) according to claim 11; characterized in that in the step of providing a sample, for which determination of species will be performed and detection of disease and pathogenicity will be realized, to a microfluidic filtration chip (102); the sample which is passed through the capillary tube with a volume of 1-2 mL and at a rate of 40 μL/minute by means of a syringe pump, a peristaltic pump or any other pump that can be used in liquid manipulation, is introduced into the microfluidic chip.

14. A method (100) according to claim 11; characterized in that in the step of providing a sample, for which determination of species will be performed and detection of disease and pathogenicity will be realized, to a microfluidic filtration chip (102); upon the sample is introduced into the microfluidic chip, filtering takes place during the liquid flow in the chip and an isolated vesicle is obtained from the outlet end of the chip, which is separated from the sample's residual materials (debris), i.e. biological molecule separation.

15. A method (100) according to claim 1; characterized in that in the step of performing determination of species, detection of disease and pathogenicity upon isothermal nucleic acid test is applied to a sample obtained from a microfluidic filtration chip (103); a strain determination is realized by transferring the isolated vesicle obtained from the microfluidic chip, to an isothermal nucleic acid amplification reaction (nucleic acid amplification reaction occurring at single-temperature such as LAMP, RPA or etc.) containing species-specific primers.
